(19) United States
(12) Patent Application Publication
(10) Pub. No.: US 2015/0191772 A1
(43) Pub. Date: Jul. 9, 2015

(54) METHOD OF CHARGING A TEST CARRIER AND A TEST CARRIER

(71) Applicant: Danmarks Tekniske Universitet, Lyngby (DK)

(72) Inventors: Andreas Hjarne Kunding, Copenhagen (DK); Sune Fang Christensen, Berkeley, CA (US)

(21) Appl. No.: 14/410,036

(22) PCT Filed: Jun. 27, 2013
(86) PCT No.: PCT/EP2013/063522

§ 371 Date: Dec. 19, 2014

(30) Foreign Application Priority Data
Jun. 29, 2012 (EP) 12174368.6

(51) Int. Cl. C12Q 1/68

(52) U.S. Cl. CPC C12Q 1/6806

(57) ABSTRACT

A method of charging a substrate with a plurality of through-going bores and a charged substrate, where the substrate is charged with a liquid comprising particles in a concentration resulting in a high percentage of bores charged with liquid with only a single particle therein.

The present invention relates to a method of charging a test carrier and a test carrier, where the test carrier has a number of bores in which a liquid with particles are introduced, and in particular a swift and simple method of providing a test carrier in which a single particle is provided in a sufficient number of the bores.

Prior art in this area of the technology may be seen in: US2011/0294678, WO03/058199, WO00/04382, WO00/22425, WO2004/090168, WO01/59432, WO2004/074818, WO01/61054, WO02/059372, WO2010/085275, US2003/0180191 and WO2011/160430.

A first aspect of the invention relates to a

A method of charging a test carrier with a liquid comprising particles, wherein the carrier comprises a slab having a number of through-going bores extending from a first side of the slab to a second, opposite side of the slab, the bores having a radius of R and a depth L, the test carrier comprising:
a first flow channel comprising a first end having a first opening, a third end having a third opening, and a first surface, positioned between the first and third ends and being at least partly defined by the first side of the slab, and
a second flow channel comprising a second end having a second opening, a fourth end having a fourth opening, and a second surface, positioned between the second and fourth ends and being at least partly defined by the second side of the slab,
   the method comprising:
adding, via the first flow channel and to the bores of the carrier, a liquid comprising a carrier liquid with a liquid/air contact angle of γ and a concentration C of particles, where the concentration C of particles and the radius R fulfill the equation of:

P₁=πCR²Lexp(−πCR²L) where L≧R cos(γ) and P₁ exceeds 0.1
flowing a first fluid through the first flow channel and
flowing a second fluid through the second channel.

The equation above, as well as any other equation in this document, should be calculated using a consistent unit-system, e.g. the SI system. It is worth noting that P1 is a probability and hence unit-less. Consequently, if the SI-system is applied, then R and L shall be expressed in units of meters, whereas C shall be expressed in units of particles per cubic-meter.

In the present context, “charging” means that the test carrier is provided with and/or supplied with the liquid.

In this context, a particle may be a biological particle, such as a cell, a gene, a protein, a DNA, a DNA fragment, an antigene, a polypeptide, an oligonucleotide, a virus, a nanoparticle or a chemical compound. Alternatively, the particle may be a non-biological particle which still is desired dosed to the bores for analysis thereof or for use in an analysis.

A slab is a flat element having a thickness, which is much lower, such as at least 2 times smaller, preferably at least 10 times smaller, than a width and length thereof.

The material of the slab may be selected in accordance with the desired use and liquid. Usually, it is desired that the slab material is not dissolvable in the carrier liquid. For certain uses and certain types of detection (see further below), it may be desired that the slab is made of a translucent material, where translucent is at a desired detection wavelength, such as IR, NIR, visible light, UV and/or X-ray radiation. Other parameters may be the surface thereof in the bores or in the vicinity of the bores, such as the contact angle of the carrier liquid thereon.

The through-going bores preferably have the same size, which is optimized to allow the liquid to span the diameter thereof while adapting the volume thereof to the concentration of particles to provide a predetermined number, such as one, of particles in each bore in as many bores as possible. Naturally, variations will under all circumstances occur due to production imperfections, so a radius and/or depth/length variation of at least 10% should be accepted.

The relationship between the volume of the bores and the volume of the loaded particles define to what extent the test carrier can be efficiently loaded. If the bore volume is smaller than the particle volume, no loading will take place. If the bore volume is much greater than the particle volume the equation for P₁ will govern the loading efficiency. Furthermore, if the volume of the bore is only slightly greater than the volume of the particle, such that only one particle can fit into the bore, then the expression for P₁ will not provide the optimal relationship between particle concentration and bore volume. The greater the bore-to-particle volume ratio gets the more accurately P₁ will predict the optimal loading concentration. An acceptable bore-to-particle volume ratio would be at least greater than 2.

Preferably, the bore volume is at least 10 times, such as at least 20 times, preferably at least 50 times, such as at least 100 times as large as the particle volume. In some situations, a bore volume to particle volume may be selected as high as 100.000 or 1.000.000 or more. In this respect, where the particle could be a DNA molecule, the radius of DNA in a solution may be estimated using the Stokes-Einstein relation:

R=k_{B}T/6πηD

where R is the hydrodynamic radius of the DNA, k_{B} is Boltzmanns constant, T is temperature, his the viscosity of the solvent and D is the diffusion coefficient of the DNA.

The diffusion coefficient relates to the length L of the DNA from the following approximation: D=Lⁿ, where n is approx. 0.571 for linear DNA and approx. 0.589 for cirkular DNA. This may be seen in more detail in (Robertson et al. Proc. Natl. Acad. Sci. USA, 2006, 103, pp. 7310-7314).

Preferably, the bores have the same diameter along their lengths, but embodiments exist in which the diameters vary in order to better hold the liquid in the bore during e.g. use or transport, or during introduction of liquid therein or during evacuation thereof.

The number of bores and the relative positioning thereof in the slab may be selected for a number of purposes. In order to obtain as many bores as possible, a small distance there between may be desired. However, when it is desired to prevent liquid contact between individual bores, a certain distance may be desired in order to prevent liquid from bridging from one bore to the other.

The carrier liquid may in principle be any type of liquid, such as water, buffer solutions, oil, organic solvents, aqueous solvents or biological fluids, such as saline, blood plasma, blood or the like.

In this context, the liquid/air contact angle (γ) is a function of the interfacial interaction energies of the three materials defining a filled bore; the liquid, the solid material of the inner walls of the bore and the air surrounding the entire slab. The contact angle follows from Young's equation as

$$cos ( γ ) = \frac{{F}_{SG} - {F}_{SL}}{{F}_{LG}}$$

where F_{SG}, F_{SL} and F_{LG} are the surface tension at the solid/air interface, the solid/liquid interface and the liquid/air interface, respectively.

The liquid comprises a concentration (C) of particles. This may be a single particle present in a concentration C, or a plurality of particles 1 . . . n, present in concentrations, C1, C2 . . . Cn, respectively, where the sum of C1+C2+ . . . Cn=C.

Fullfilling the equation with P1 exceeding 0.1 means that at least a predetermined proportion of the bores comprise one of the particles.

Preferably, P1 exceeds 0.2, such as exceeds 0.25, preferably exceeds 0.3, such as exceeds 0.35.

In one embodiment, the adding step comprises flowing part of the liquid from a first side of the carrier, through the bores and to another side of the carrier. In this manner, it is ensured that a sufficient amount of liquid is introduced into the bores and that no air bubbles are present therein which would otherwise take up space and thus reduce the amount of liquid in the bore. This embodiment may be obtained by using the liquid flow-paths on both sides of the slab so that one flow path may be used for introducing liquid to the slab and the other may be used for receiving liquid exiting the bores.

Subsequent to this adding step, fluids is/are flowed through the first and second flow channels. This subsequent step may be performed to prevent fluid connection or bridging between bores—at least the fluid of the first liquid. This is especially interesting when different reactions take place, see further below, in different bores, where reaction products could otherwise flow from one bore to the next and thus “pollute” the contents of the next bore. This is especially a disadvantage when the contents of the bores differ and when a subsequent step (see further below) aims at detecting or determining reactions taking place or having taken place in the bores.

This preventing of liquid connection may be obtained by evacuating or drying the surface(s) of the slab. Blow drying could be used, whereby the first and/or second fluids may be air or gas. Evaporation of liquid is also a possibility, whereby the first and/or second fluids may also be air or gas. Additionally or optionally, surface parts of the slab at least in the vicinity (such as the area closer than R to the bore) of the bore may be hydrophobic, such as covered by a hydrophobic material.

Especially in the situation where the flow paths are provided, removing liquid in the flow path may be performed by forcing air through the flow path. Both in this situation and the situation where a liquid is used for removing excess liquid, this may enter a flow path through one opening and exit through another opening so that air, liquid or gas forced into the flow path will evacuate the flow path or at least remove liquid from the slab surface, without forcing the liquid out of the bores.

If one of the first/second fluid is a liquid, preferably immiscible liquids should be utilized. Hydrophobic and hydrophilic liquids are in most cases immiscible, e.g. water with oil, but immiscibility can also be induced by external changes in such as temperature and pressure or by internal changes such as the molar ratios of the first/second liquids. Commonly, miscibility can be deduced from a liquid/liquid phase diagram calculated on the basis of the Gibbs energy of mixing between said liquids.

Naturally, the liquid/fluid/air/gas used for flowing in the first and second flow paths may be the same or different. If the liquid/fluid/air/gas is the same, the same reservoir and/or pump may, of course, be used.

In one embodiment, the method further comprises the step of, subsequent to the adding step, fixing one or more of the particle(s) in at least one of the bores, preferably all bores or substantially all bores in which one or more particles exist. Fixing may take place by direct covalent attachment using well-known chemical reaction partners such as maleimide-groups reacting with thiol-groups, amine-groups reacting with carboxyl-groups and azide-groups reacting with alkyne-groups. Here, the element/particle can display any of the mentioned groups and the inner surface of the bore can display any of the complementary chemical groups. Fixing may also take place by strong non-covalent interactions between ligands/receptors, antigens/antibodies, haptens/antibodies or gold/sulphur.

Having thus fixed the particles, a number of advantages are obtained and a number of steps may now be performed without removing the particles from the bores.

In one situation, the method may be followed by, subsequent to the fixing step, a second step of adding the liquid to the bores. Thus, bores in which no particle(s) were received during the first step of adding the liquid, may receive particle(s) during the second step, so that the overall number of bores with one more particles may be increased by the second step. This re-charging of the slab may act to increase P1 from the value obtained during the first step to an even higher number.

In that or another situation, the method may further comprise the step of evacuating carrier liquid from the bores, preferably without removing the fixed particles. Then, other liquids, fluids, substances, agents, particles or the like may be introduced into the bores.

This evacuation may be performed by replacing the carrier liquid with another liquid or with air or gas. Thus, another liquid or a gas may be introduced into the bores and may be forced or pumped there into in order to remove or replace the carrier liquid.

Additionally, the below-mentioned amplification/multiplication step may be performed wherein a particle in a bore is multiplied or amplified (copied) within the bore.

A second aspect relates to a method of charging a test carrier with a liquid comprising particles, wherein the carrier comprises a slab having a number of through-going bores having a radius of R and a depth L, each of a first plurality of the bores comprising one or more elements each operative to fix a particle to the bore,

the method comprising:
1. adding, to the bores of the carrier, a first liquid comprising a carrier liquid with a liquid/air contact angle of γ and a concentration C of the particles, where the concentration C of particles and the radius R fulfill the equation of:

P₁=πCR²Lexp(−πCR²L) where L≧R cos(γ) and P₁ exceeds 0.1,
2. the fixing elements, in each of a second plurality of the bores of the first plurality, fixing one or more of the particle(s) of the liquid added to the pertaining bore,
3. amplifying/multiplying the particle(s) in the second plurality of bore(s),
4. adding the first liquid to at least one of the bores of the first plurality but not being within the second plurality of bores,
5. the fixing elements, in each of a third plurality of the bores not being part of the second plurality of bores, fixing the one or more of the particle(s) of the liquid added to the pertaining bore,
6. amplifying/multiplying the particle(s) in the third plurality of bore(s),

In this aspect, all comments made in relation to the first aspect are also relevant.

According to the second aspect, the above-mentioned fixing of the particles takes place, whereafter a multiplication/amplification/copying step is performed. This step has multiple advantages. One advantage is that when more particles are present in the bore, a more easily detectable presence may be obtained. Another advantage is that by multiplying the number of particles in the bore, all fixing elements of the bore may be utilized or taken up so that the subsequent step of adding the first liquid may result in the adding of liquid—and new particles—to the bore, but such particles will not be able to fix to the bore and thus may be removed prior to a detection step or the like.

The aspects of the invention may be combined if desired. Thus, the purging/cleansing steps of the first aspect may be used in the second aspect in order to e.g. prevent contents of one bore from contaminating a neighboring bore.

In this respect, the amplifying/multiplying step preferably is performed as a template-directed synthesis, such as a synthesis in which the one or more particle(s) serve as input template(s), and where the number of fixing elements per bore of the second plurality is adjusted to be less than the total number of reproduced particles, such that reproduced particles undergoing fixation eliminates or takes up all fixing elements in the said second plurality of bore(s),

This step may be performed by adding a second liquid to the second plurality of the bores containing elements for triggering template-directed synthesis, where the particles constitute the template.

In this respect, the amplifying/multiplying step preferably is performed such that if N_{c}(t) is the number of copies produced after a given time t during the multiplication step, and N_{FE} is the number of fixing elements per bore, then the minimum duration of the step should be chosen such that N_{c}(t)>N_{FE}. As is known to those skilled in the art, a template-directed multiplication reaction will usually proceed exponentially over time, such that N_{c}(t)=α^{t}, where α is a positive number greater than 1, and usually between 1 and 2. In this case, the minimum duration of the multiplication step can be estimated as t=In(N_{FE})/In(α).

During step 1, particles are added to a number of the bores, i.e. the second plurality of the bores. However, statistically, a number of the bores will receive no particles, whereby step 2 will not result in fixing of particles and step 3 will result in no multiplication of particles therein.

Step 4 may be preceded by an evacuation of liquid in the bores in order to facilitate replacement or adding of liquid and particles to the bores.

In step 4, particle(s) may be added to bores which received no particles in step 1. Thus, statistically, step 4 will result in particles being present in more bores than after step 1.

Naturally, step 4 may also comprise adding a particle to a bore already having a particle, but if step 3 has, as is preferred, resulted in all fixing elements of that bore being taken or occupied, such particle is not able to fix to the bore and thus may be flushed out of the bore.

Step 5 may be identical to step 3 in that the same process may be performed.

Naturally, steps 1 and 4 may comprise providing the sample to all bores, but the liquid in the steps may be selected differently, such as with different types of particles or liquid, if desired.

Also, steps 3 and 5 may be performed for all bores or for different bores if desired. These steps may be performed adding other liquids/fluids to the bores as well as performing other steps, such as tempering or the like, facilitating the amplification/multiplication/copying desired. This process may be targeted some types of particles so as to amplify/multiply some types of particles in the sample and no others.

A third aspect of the invention relates to a method of using a test carrier, the method comprising:
providing a test carrier according to the first or second aspects of the invention,
adding, to the bores, a second liquid comprising one or more first substances,
detecting a reaction between one or more of the one or more first substances and one or more of the particles.

The addition of the second liquid may be performed using the above flow paths which may be used for guiding the second liquid.

In this aspect, the test carrier may be used for testing the second liquid or the one or more substances. These one or more substances may be cells, virus, virus-like particles, genes, proteins, polypeptides, oligonucleotides or optical probe elements.

The reaction may be any form of reaction between the particle(s), or at least, if the liquid has multiple types of particles, one of the particle types, and the second liquid, such as the first substance(s).

The reaction may be oligonucleotide amplification reactions, ligand binding assays, enzyme activity assays, in vitro oligonucleotide transcription, in vitro oligonucleotide expression, restriction endonuclease reactions, protease reactions or kinase reactions.

The detection of the reaction may be a detection of the presence or absence of a product, such as a particle type, a substance, a liquid or the like. The detection may be a quantification of a concentration or a number of elements, such as particles, in individual bores, or the quantification may be a quantification of a number of bores in which a detection is positive or negative, such as the bores in which a quantification of the presence or absence of a substance is determined, such as where a concentration of the substance is compared to a threshold value, where the substance may be determined as absent, if the concentration thereof is below a threshold, or where the substance may be determined as present, if the concentration thereof exceeds a threshold.

The detection may be performed in any of a large variety of manners. The detection of fluorescence of a substance generated by the reaction may be determined. Alternatively, the absorption or scattering of a substance generated or consumed by the reaction may be determined. Detection of other forms of signals may be nuclear magnetic resonance, radioactivity or surface plasmon resonance.

When an optical detection is performed, the slab with contents (particles and liquid in the bores) may be removed from e.g. the flow paths described in order to allow optical access to the contents of the bores. Alternatively, the flow paths and other elements potentially positioned between the contents of the bores and a detector, and potentially a radiation source, may be provided of a material translucent at the desired wavelength or wavelength interval. Additionally, the slab itself may be made of a radiation translucent material if desired.

In one form of detection, contents of each bore is removed individually and analyzed in an analyzer suitable for the purpose. These contents may be a liquid of the bore in which a substance to be quantified may be present or absent. It is noted that the actual types of processes performed in the bores may be all types of processes known today, and that the analysis performed on the contents of each individual bore may be that known today. The advantage being that a large number of parallel processes and detections is possible.

The advantage of especially optical detection is that this detection may be performed simultaneously in a plurality of bores. This detection may be based on a picture taken by a camera viewing a plurality of the bores, such that individual detection of each bore is avoided.

Naturally, the method may further comprise the step of, before or after introducing the second liquid but before the detection step, performing additional operations on the particles, carrier liquid and/or the second liquid.

In a preferred embodiment, the particles are DNA fragments, where a PCR procedure may be used to multiply these while in the bores. Where the fragments are fixed or tagged, tagged primers may be used to also ensure that the generated particles or DNA fragments are fixed to the bore wall.

This additional step may also be a step of incubation, heating, shaking, irradiating the slab and/or its contents or the like.

In a preferred embodiment, the method further comprises the step of fixing one or more of the particle(s) in at least one of the bores, where the adding step comprises replacing at least part of the carrier liquid in the at least one of the bores while the particle(s) therein is/are fixed.

This fixing of the particles has a number of advantages.

Firstly, as is also mentioned above, multiple steps of introducing the first liquid may be performed to increase the number of bores in which the desired (number of) particle(s) is/are present.

Secondly, as mentioned above the fixing may be used for maintaining the particles in the bores while replacing e.g. the carrier liquid therein. In this manner, the carrier liquid may be removed while ensuring that the particles stay. In this manner, the volume of the second liquid in the bores may be known, as it may fully replace all carrier liquid in the bores, or at least it may be present in a sufficient concentration to ensure that the reactions in the bores take place as desired.

Thirdly, the fixing may be used for performing multiple tests using the same test carrier. Now that the particles are fixed, the used test carrier on which the detection has been performed may have the liquid in the bores replaced by a third liquid with a second substance. The third liquid and the second substance may be identical to or different from the second liquid and the first substance. Identity means that the same process is repeated and that an additional detection may be made. Knowing that the particles fixed remain in their bores, a the first and subsequent detections may by compared in that even though the contents of the bores may differ (one bore may have a particle, one may have multiple, one may not have a particle, two different bores may have different particles etc), the subsequent detections may rely on the particle-contents of a bore to be maintained.

Thus, a reaction detected in a bore using the second liquid may be compared, bore for bore or bore position for bore position, to a subsequent detection based on the same second liquid or a reaction seen in a subsequent detection using a third liquid.

A fourth aspect of the invention relates to

test carrier with a liquid comprising particles, wherein
the carrier comprises a slab having a number of through-going bores, the bores having a radius of R and a depth L,
the liquid comprises a carrier liquid with a liquid/air contact angle of γ and a concentration C of particles,
   where the concentration C of particles and the radius R fulfill the equation of:

P₁=πCR²Lexp(−πCR²L) where L≧R cos(γ) and P₁ exceeds 0.1,

the test carrier further comprising a first and a second flow channel, the first flow channel comprising a first end having a first opening and a first surface at least partly defined by a first side of the slab and opening into the bores, the second flow channel comprising a second end having a second opening and a second surface at least partly defined by a second side of the slab and opening into the bores.

As mentioned above, P1 preferably exceeds 0.15, such as exceeds 0.2, preferably exceeds 0.25, such as exceeds 0.3, preferably exceeds 0.35.

All circumstances and comments made in relation to the first, second and third aspects of the invention are equally valid here, such as that the concentration C may be composed of concentrations of a number of particles present in the carrier liquid.

In one embodiment, the bores have a hydrophilic surface and the slab has, in areas in the vicinity of the bores, a hydrophobic surface. In this context, a “hydrophilic” surface is a surface, which induces contact angles with water less than 90°, at the water/air interface. A “hydrophobic” surface is a surface, which induces contact angles with water greater than 90° at the water/air interface. The “vicinity” of the bores may be areas within a predetermined distance from an edge to a bore, such as ½, 1, 2, 3, 4, 5, 6, 7, 8, 9 or 10 times R, for example.

The hydrophobicity and/or hydrophilicity may be caused by the material of the slab itself or by a coating. A generally hydrophilic material is e.g. silanes, silicon oxides or metal-organic compounds and a generally hydrophobic material is e.g. alkanes, fluorinated alkanes or carbon.

The above embodiment relates primarily to situations where the carrier liquid comprises water. In other situations, where the carrier liquid comprises buffer solutions, oil, organic solvents, aqueous solvents or biological fluids, such as saline, blood plasma, blood or the like, the relevant parameter is the contact angle between the surface of the bores and the surface in the vicinity of the bore openings.

The first and second sides of the slab preferably are the opposite, flat sides thereof having openings into the bores.

The flow channels may be formed by one or more elements combinable with the slab to form the flow channels and the relationship between the flow channels and the bores.

In a preferred embodiment, the material of these elements is translucive at least at a desired wavelength or wavelength interval so that an optical detection may be performed of contents in the bores without having to remove the elements from the slab before detection.

In general, “translucive” means that the transmission of the material is at least 25%, so that at least 25% of the radiation impinging on the test carrier at a predetermined wavelength is allowed to pass without absorption.

Naturally, translucence may be desired differently from the elements forming the two flow channels, as radiation at one wavelength may be desired launched to the bores from one direction and radiation at another wavelength emitted from the bores may be desired detected from another direction.

In a preferred embodiment, the flow channels each have two openings and a channel there between where a surface part of the channel is formed by a side of the slab. Thus, the first flow channel may further comprise a third end having a third opening, the first surface being positioned between the first and third ends and the second flow channel may comprise a fourth end having a fourth opening, the second surface being positioned between the second and fourth ends.

In this manner, a liquid or gas may be provided though the flow channel without having to be forced through the bores. In one situation, this may be used for evacuating the flow channels and thus prevent liquid contact from bore to bore, as is described above.

In one embodiment, as is also described above, the test carrier may comprise one or more elements operative to fix the particle(s) in the bores, such as the methods described above.

In this relation, the below manner of temperature controlling the slab using tempered, humid gas may be used.

A fifth aspect of the invention relates to an apparatus for performing an analysis, the apparatus comprising:
a test carrier according to the fourth aspect,
a sample supply operational to feed a sample to the bores of the test carrier,
a supply of first and second fluid operational to feed first and second fluid to the first and second flow channels, respectively, and
an analysis element operational to detect or determine a reaction between one or more of the particle(s) of the bores and the sample.

In this context, the sample may be a liquid, fluid, gas or the like. The sample may comprise a substance, as is also described further above.

The sample supply may comprise a container for holding the sample or an element operable to remove sample from a sample container, such as a tube, connection or the like and feed it to the test carrier. Also, the supply may comprise a pump for forcing sample from the container/tube and to the test carrier.

The test carrier may be provided inside an enclosure in order to better provide the liquid therein/on and/or so as to protect the carrier from pollution/evaporation.

The analysis element may be adapted to perform any of the above-mentioned types of analysis, such as a photo/radiation detector. Also, the analysis element may comprise a radiation emitter, if e.g. an absorption/scattering method is used or if radiation is required to facilitate the reaction or to facilitate emission of lower wavelength radiation from the reaction or a substance or reaction product thereof.

In one embodiment, the sample supply comprises a pump for pumping the sample via the first flow channel and the first opening.

Naturally, the first or second flow channel may be used for introducing the liquid to the test carrier. If no other openings are provided, a liquid flow through the first flow path takes place through the bores and into/from the second flow path.

The supply of first and second fluid may be configured to supply the first/second fluid to evacuate the first and/or second flow channels for liquid or at least the first liquid.

The first and second fluids may be the same fluid, so that a single pump or the like may be used. Different types of fluids are described above.

The two openings of one flow channel may be used also for, as described above, evacuating the flow channel so as to prevent liquid communication between bores. This evacuation may be desired on both sides of the test carrier, and to that effect a gas pump, such as an air pump, may be provided for generating the desired gas/air flow in one of or both first and the second flow channels.

As mentioned above, in one situation, the sample is operational to generate, when reacting with predetermined particle(s), an optically detectable result. In this situation, the analysis element may be as described above. Also, it may be desired to not remove the slab from any other elements, such as elements forming the flow channels, whereby such elements preferably are translusive at the desired wavelengths or wavelength intervals.

It may further be desirable to ensure temperature controlling the substrate. This may be obtained by feeding a temperature controlled gas comprising a predetermined amount of a second liquid to the substrate. Here comprising refers to the absorption of a second liquid by evaporation into the temperature controlled gas.

The overall aim is to temperature control the substrate with the first liquid using the gas and second liquid, preferably while the presence of the second liquid in the gas prevents or reduces evaporation of the first liquid from the openings.

In a preferred embodiment, the substrate and the first liquid therein is for use in an analysis of the first liquid or particles, such as those mentioned above. According to this aspect, the substrate may have through-going bores, as described above, or cavities holding the first liquid. The first liquid may itself fill the openings, or other elements, such as the above particles, may also be present.

In a simple situation, the first and second liquids are the same. This will typically be the situation where the first liquid is a single substance or material, such as water, or a liquid comprising dissolved substances, such as salts, which are not prone to evaporation in the temperature range of interest.

If the first liquid present in the openings is a mixture of multiple substances or liquids, these different substances or liquids may have different boiling temperatures, evaporation temperatures or the like and may thus see different amounts of evaporation or other type of escape from the first liquid and the openings over time.

Thus, the composition of the second liquid in the gas may deviate from the composition of the first liquid, even though, usually, the components or liquids/substances of the second liquid will be the same as those of the first liquid, but the first liquid may comprise additional liquids/substances not present in the second liquid. Usually, the second liquid will comprise those liquids or substances of the first liquid which are the most prone to evaporation. The relative concentrations of such liquids or substances preferably reflects the evaporation of these liquids/substances, so that a higher amount of a liquid/substance is seen, if this liquid/substance is more prone to evaporation.

The openings may be through-going bores, as described in relation to the above aspects of the invention, or may be cavities.

In one embodiment, the gas is fed to a flow channel connected to the surface of the substrate. This has the advantage that the interaction between the first liquid and the gas and second liquid is better controlled.

Preferably, the amount of the second liquid in the gas ensures that at least substantially no loss of the first liquid is seen in the openings. As mentioned above, if the first liquid comprises multiple liquids, this loss may be controlled and counter-acted on a liquid-by-liquid basis where the relative amounts of such liquids—present in the first liquid—in the second liquid will act to ensure that no net evaporation or loss of that particular liquid is seen in the openings.

It is noted that the degree of evaporation of different liquids may differ with temperature. Thus, when the temperature changes, different amounts of the second liquid may be desired in the gas. Also, if the second liquid comprises multiple different liquids, different relative amounts of these liquids may be desired or provided depending on the temperature.

In this respect, the gas may be selected in accordance with a number of parameters. Normally, it is desired that the gas does not interfere with the first or second liquids or particles or the like therein. Thus, it may be desired that the gas has no oxygen, if oxidization could be a problem in the liquids or particles. Ambient air may be used in many instances. Nitrogen, helium, neon or argon may be used in the same instances or in instances where oxidization may be a problem.

Then, an apparatus for providing a temperature controlled environment may be derived, the apparatus comprising:
a substrate comprising a surface having a plurality of openings each holding a liquid,
a gas provider providing a temperature controlled gas, comprising a predetermined amount of the liquid, to the surface.

As mentioned above, the substrate may be as that described in the first aspects of the invention or a substrate having openings in stead of through going bores.

In one situation, the apparatus, further comprises an element forming, with the surface, a flow channel guiding gas from the gas provider to the surface. If the substrate has through-going bores, a flow channel is preferably provided at both surfaces of the substrate having openings into the bores. Preferably, the same gas and second liquid is fed to both channels.

In one situation, as is also described above, the gas provider comprises a liquid supplier adapted to supply the predetermined amount of the second liquid to the gas.

As mentioned above, different liquid supplies may be desired if the second liquid comprises different liquids and especially when the relative amounts of the different liquids will depend on the temperature.

The temperature controlling may be performed in any desired manner, such as providing the gas and/or the second liquid through a heat exchanger, a heating coil, or the like.

The gas provider may comprise a pump. Alternatively, the gas may be provided in a pressurized container, the output flow of which is used for driving the gas and second liquid to the substrate surface.

The second liquid preferably is provided to the gas as a gas itself. Thus, an evaporator may be provided which evaporates the second liquid and feeds it to the gas as an evaporated liquid.

In the following, preferred embodiments of the invention are described with reference to the drawing, wherein:

### BRIEF DESCRIPTION OF FIGURES

FIG. 1: Example graph of the optimal relationship between concentration and bore volume.
A. 2D plot of Eqn. 3 with γset to π/4.
B. Sketch of a bore cross-section with R, L and γ indicated.
C. Sketch of a bore with L close to its minimum value.

FIG. 2: Sketch of two embodiments of the apparatus.
A. The upper and lower flowchannels are operated by pushing the fluid through.
B. The upper flowchannel is operated by pushing the fluid through, whereas the lower flowchannel is connected to a vacuum.

FIG. 3: The minimal number of components to assemble the apparatus.
A. Top part of the flowsystem with inlets/outlets and holes for alignment.
B. Upper flowchannel.
C. Chip comprising an array of bores.
D. Lower flowchannel.
E. Bottom part of the flowsystem.

FIG. 4: Example of loading the array
A. An unloaded array.
B. Fluid 1 is introduced and fills out the bores.
C. Fluid 2 is added and dispels fluid 1 from the outer faces of the array.
D. The bores containing fluid 1 are now immersed in fluid 2.

FIG. 5: How to carry out confined assays inside the bores of the array.
A. A receptor molecule is attached inside a bore.
B. A solution of ligands is added to the array.
C. Ligands bind to the receptors.
D. Excess ligands are removed by flushing the array.
E. The bore only contains receptor/ligand-complexes.

FIG. 6: An embodiment of the apparatus adapted for parallel optical measurements comprising an illumination source (top), the flowsystem (center) and an imaging detector (bottom).

### MODES FOR CARRYING OUT THE INVENTION

Optimal Loading Conditions.

For many applications, it is desirable to confine a single biological element and subsequently subject it to various analyses. These include, but are not limited to, digital polymerase chain reaction (PCR), single molecule profiling and spectroscopy, preparation of mutant oligonucleotide libraries, single molecule enzyme-linked immuno-sorbent assay (ELISA) and for measuring the immunological activation of single cells. Some of these analyses are carried out with the aid of flow-cytometric instrumentation, thus enabling single analytes to pass by a detector one at a time. However, in that way the measurement duration will scale linearly with the number of elements being analyzed. Consequently, a measurement on a large number of analytes will greatly benefit from arraying individual analytes on a surface, thus rendering the measurement compatible with e.g. imaging-based and/or surface-based high throughput analyses.

In order to produce an array exhibiting the highest fraction (P₁) of loaded array sites (here loaded refers to an array site with exactly one biological element inside) the concentration of elements (C) has to be adjusted according to the volume (V) of each bore. The relationship between C and V follows from the Poisson distribution and is given below:

P₁=CVexp(−CV) Eqn. 1

However, V is not free to assume any value, since it is constrained by the geometry of the bore. For example, if the bore is too shallow, surface tension will not be sufficiently strong to maintain a liquid film across the entire bore cross-section. For a bore of inner radius (R) and depth (L) containing a liquid forming a contact angle (γ) with the inner side of the bore the minimum value of L is

L=R cos(γ) Eqn. 2

Consequently, since V=πR²L we transform Eqn. 1 to

P₁=πCR²Lexp(−πCR²L), L≧R cos(γ) Eqn. 3

Array Description and Fabrication.

In one embodiment of the invention, the slab 16 (see FIGS. 1 and 2) comprises a high-density array of bores 12, where the inner wall 22 of each bore 12 is rendered hydrophilic and the outer faces 24 of the slab 16 are rendered hydrophobic. Preferably, the hydrophilic material is composed of glass (e.g. SiO₂) and the hydrophobic material may be a coating of fluorinated carbons (e.g. C₄F₈). This configuration triggers spontaneous droplet formation inside the bores, renders each bore fluidically insulated from its neighbors and by virtue of the SiO₂ provides a suitable material for further biofunctionalization of the inner surfaces of the bores. Examples of functionalization methods include silanazation, physisorption of biomolecules/polymers and self-assembled molecular mono- and bi-layers.

A slab meeting the specifications as the ones described above can be fabricated by a person skilled in the art of microfabrication, e.g. photolithography, deep reactive ion etching, surface layer deposition, wet etching and imprint lithography. For example, using photolithography a pattern of circles with diameters down to 1 μm can be produced in a thin film of photosensitive material added onto a silicon substrate. Next, the pattern can be used as a mask for anisotropic etching of the bores into the silicon substrate using deep reactive ion etching. The depth of the bores can subsequently be adjusted by isotropic etching using, e.g. potassium hydroxide. A thin layer of glass can be grown by thermal oxidation of the substrate. Since, glass is optically transparent, whereas silicon is not, this design would reduce optical cross-talk between bores during the detection/read-out process. Finally, a hydrophobic coating of e.g. C₄F₈ can be applied by masking the substrate with a photoresist followed by plasma-deposition of C₄F₈ followed by removal of the residual photoresist by a lift-off process.

Flow-System and Reagent Delivery.

An array of bores can be integrated into a suitable flow-system to enable exchange and delivery of liquids and/or gases containing suitable reagents. The flow-system can be produced from a variety of different materials including glass, plastic, elastomers or metal. In one example, the upper/lower part 32, 34 of the flow-system can be fabricated by milling of the desired structure in a plastic material such as poly(methyl methacrylate) (PMMA), se FIG. 3. The middle part of the flowsystem can be fabricated by molding of the flowchannel structure into a suitable elastomer, such as poly(dimethyl siloxane) (PDMS). An elastomeric material is preferable, since it will function as a deformable gasket, thus preventing leakage of liquid from the flowsystem, once it has been properly clamped, either mechanically or covalently.

The chip comprising the array of bores is fitted in the flowchannel and the flowsystem is sealed by clamping (see also FIG. 3) of the middle parts (and the chip) by applying pressure on the upper and lower parts of the system. The chip can now be fluidically contacted by inserting tubes 36 into the inlet/outlet of the flowsystem, thus enabling reagent delivery. The fluid flow may be enabled by connecting the inlets to pumps pushing the liquid through the channels (FIG. 2A) or by only pushing the liquid through the upper flowchannel, whereas the outlet of the lower flowchannel is connected to vacuum (FIG. 2B).

Charging of the Apparatus for Analysis/Measurements.

An apparatus (see FIG. 4), comprising an array of bores on a chip fitted into a flowsystem, as the system described above, can be charged by introducing a solution 42 of biological elements to the bore array via the flowchannels. Once, fluidic contact with the individual bores has been established, capillary forces will pull the liquid into the bores and retain them there. This process will take place when both the inner walls of the bore and the liquid are both hydrophobic or both hydrophilic. If the inner wall of the bore is hydrophobic and the liquid is hydrophilic, the liquid will not extend into the bores. The same is the case for the opposite scenario, i.e. hydrophilic inner walls of the bore and a hydrophobic liquid.

For bore volume and concentration adjusted according to Eqn. 3, a maximum number of bores will be loaded with a single biological element. The inner volume of individual bores can be rendered fluidically insulated in a number of ways following the initial loading. If the loading liquid and the bore are both hydrophilic, a second hydrophobic liquid 44 may be introduced via the flowsystem, thus displacing the loading liquid on the outer faces of the slab, while retaining loading liquid inside the bores due to capillary forces. If the loading liquid and the bores are both hydrophobic, the second liquid would have to be hydrophilic to achieve confinement of the loading liquid.

In general, the first and the second liquid should be immiscible with each other to obtain confinement of the first liquid inside the bores. Alternatively, the loading liquid can be displaced with a flow of air 44. In the case of an aqueous loading liquid, the air-flow can be saturated with water and kept at a constant temperature in order to enable rapid temperature change of the array without losing the bore-confined liquid due to evaporation.

Once an array of fluidically insulated volumes have been established a large number of applications, assays, analyses and measurements can be conducted in parallel by introducing reagents via the flowsystem in the right order. Many of these applications, assays, analyses or measurements are conventionally conducted in reaction tubes, but will benefit from being adapted to a parallel array format, since such a feature enables high throughput, multiplexing capacity and decreased reagent consumption. A number of specific applications are outlined below:

Surface-Functionalization.

Specific attachment of biological elements to the inner walls of the bores can be carried out (see FIG. 5) by a variety of different methods. For example, functionalization of the inner walls of the bores can be achieved by introducing a solution of poly-L-lysine grafted with poly(ethylene glycol) (PLL-g-PEG). As is known to those skilled in the art, PLL-g-PEG spontaneously adsorbs to silicon-oxide under mildly acidic buffer conditions, and hence specifically binds to the inner walls of the bores. Furthermore, PLL-g-PEG is commercially available (Surface Solutions, Switzerland) in various derivatized forms, where functional chemical groups have been attached on the PEG. Functional groups include carboxylic acids and amines, which can be utilized for specific covalent conjugation, and also include biotin and nitril-acetic acid moieties, which binds specifically to avidin-like proteins and polyhistidine-tagged molecules, respectively.

Furthermore, by mixing non-functionalized PLL-g-PEG with PLL-g-PEG molecules containing functional groups as the ones mentioned above, then the number of attachment sites on the inner side of the bores can be directly controlled by adjusting the stoichemetry of the mixture. For example, using a PLL-g-PEG with the specifications of PLL(20)-g[3.5]-PEG(2) yields a surface density of PEG groups between 0.2-0.5 per nm2 depending on the conditions (Pasche et al, J. Phys. Chem. B. 2005, 109, pp. 17545-17552). Hence, if one were to mix PLL(20)-g[3.5]-PEG(2) with PLL(20)-g[3.5]-PEG(2)-biotin, where the latter molecule has a functional biotin group attached on its PEG-units, in a ratio of 1:1, 1:4 and 1:10, then the following surface densities of PLL(20)-g[3.5]-PEG(2)-biotin would be 0.1-0.25 per nm2, 0.04-0.1 per nm2 and 0.02-0.05 per nm2, respectively.

Alternatively, protein molecules may be adsorbed directly onto the inner surface of the bores. This can be achieved by adjusting the pH of the protein solution, such that the proteins (depending on their isoelectric point) will become positively or negatively charged, thus creating an attractive interaction between the positively or negatively charged inner surfaces of the bore. Silicon dioxide exhibits an isoelectric point in the range from 1.7-3.5 and is thus negatively charged at physiological pH-values around 7. Hence, in the case of silicon dioxide as the constituent material for the inner bore surface, the adsorbant molecule should preferably exhibit a net positive charge. Furthermore, surfaces of silicon dioxide can be supplied with specific chemical properties by covalent modification (e.g. silane derivatization), thus introducing various functional chemical groups (carboxylic acid, amine, thiol, azide, alkyne, alcohol) at the inner surfaces of the bore.

Nucleotide Amplification.

Nucleotide amplification reactions can be carried out inside individual fludicially insulated bore-volumes. First, a loading liquid containing a solution of oligonucleotides, with concentration adjusted according to Eqn. 3, is introduced to the array as described above, thus forming individual fluidically insulated droplets captured in each bore. The majority of droplets contain only a single oligonucleotide, which will be designated as the amplification target. To enable amplification, the loading liquid also should be supplied with proper oligonucleotide primers as well as a mixture of molecular components assisting the amplification. Depending on the particular amplification-mixture, the reaction may proceed via different mechanisms, i.e. polymerase chain reaction, ligase chain reaction, rolling circle amplification, helicase assisted amplification or recombinase polymerase amplification. Some of these amplification reactions require thermal-cycling to proceed, whereas others take place at a constant temperature. In both cases, the desired temperature sequence can be achieved by flowing a stream of temperature-controlled humidified air over the outer surfaces of the array. When the air is humidified it will impede solvent evaporation, as induced by changes in temperature.

The amplification products may be attached to the inner surfaces of the bore by applying a surface-functionalized bore and a chemically tagged oligonucleotide primer. For example, a bore functionalized with PLL-g-PEG, where the PEG displays a biotin moiety bound to an avidin-like molecule, would be able to capture biotin-tagged amplification products. A biotin-tag may be incorporated in the amplification products by using a biotin-tagged oligonucleotide primer. Alternatively, reactive chemical groups (carboxylic acid, amine, thiol, azide, alkyne, alcohol) can be incorporated into the amplification products using modified primers, thus facilitating covalent attachment of the oligonucleotides on the inner surfaces of the bore by reacting with complementary groups. As another alternative, the amplification products may be attached to the inner surfaces of the bores by prior functionalization of the surface with peptide nucleic acids (PNA). PNA is commercially available with various reactive chemical groups to enable surface-attachment and is able to recognize and bind specific oligonucleotide sequences situated on the amplification products.

If the number of attachment sites is smaller than the number of amplification products, then the inner surface of the bore will become saturated with bound amplified oligonucleotides. In this way, if oligonucleotides are subsequently introduced to the bore, there will be no sites available for attachment for them. Consequently, it can be ensured that once a single amplification reaction has taken place within the same bore, and has been allowed sufficient time to populate all available attachment sites, then no subsequent amplification reaction will be able to make use of the attachment sites, because they are already occupied. This is an advantage for increasing the number of loaded bores, by repeatedly executing the following sequence of actions; (i) charge the bores with an oligonucleotide solution according to Eqn. 1, such that the number of bores with only one oligonucleotide present is optimal (ii) multiply the single oligonucleotides to several copies, which attach and saturate on the inner side of the bore and (iii) flush all the bores to remove excess unbound oligonucleotides. In the case of loading the array with oligonucleotides of the same sequence, the array may be useful for various genetic analyses, including digital polymerase chain reaction. In the case of loading the array with oligonucleotides of differing sequences, the array may be useful for preparation of surface-immobilized gene libraries.

Nucleotide Manipulation.

A variety of tools to manipulate and modify oligonucleotides are known to those skilled in the art, including, but not limited to, oligonucleotide cleavage facilitated by restriction endonucleases, covalent attachment of two or more oligonucleotides facilitated by ligases, transcription of oligonucleotides to messenger ribonucleic acids using in vitro transcription kits, expression of polypeptides from oligonucleotides using in vitro translation kits, reverse transcription of oligonucleotides facilitated by reverse transcriptase and hybridization of oligonucleotides with oligonucleotide detection elements, for example fluorescence in situ hybridization.

In all the cases, a reaction mixture containing the required chemical components is introduced to the array and subsequently removed to form individual bore-retained droplets, as described above. The reaction mixture can either be introduced simultaneously with a loading liquid consisting of oligonucleotides or it can be introduced alone to a pre-loaded array hosting oligonucleotides retained inside the bores. The temperature can subsequently be adjusted depending on the conditions required for the assays to be carried out. Following completion of the assay, the array may be flushed with a third liquid to remove undesired products or unreacted reagents from the bores. Depending on the type of assay, the progress of the reactions may be monitored while they take place in the bores using imaging-based detection.

Polypeptide Expression.

Standard protocols for expression of polypeptides from oligonucleotides may be performed in a parallel format inside individual bores using the described apparatus. A reaction mixture containing all the required chemical components for enabling the production of polypeptides from oligonucleotides can be introduced to the array via the flowchannels and subsequently removed from the outer surfaces of the slab, as described above. The reaction mixture can be introduced at the same time as a loading liquid containing oligonucleotides or it may be introduced to a pre-loaded array. Polypeptides produced in this way may be attached inside the bores by utilizing a proper combination of chemical or biochemical tags, for example by incorporation of a polyhistidine tag into the polypeptide sequence and a nitril triacetic acid group on the bore surface. Polypeptides with unnatural amino acids incorporated in their sequence may be produced in a similar way, but by supplementing the reaction mixture with any desired unnatural amino acids, and by insertion of the associated translation codon in the template oligonucleotide.

Polypeptide Manipulation.

Several assays to manipulate and modify polypeptides are available to those skilled in the art, which may be conducted inside individual bores of the array. These include proteolytic degradation of polypeptides using enzymes, chemical or biochemical labeling of specific residues of the polypeptide, capture or binding of the polypeptide by antibodies and enzymatic addition/removal of functional chemical groups on the polypeptide, for example phosphorylation, ubiquitinylation or glycosylation. The reactions may be carried out and be adjusted as explained above.

Parallel Optical Analysis.

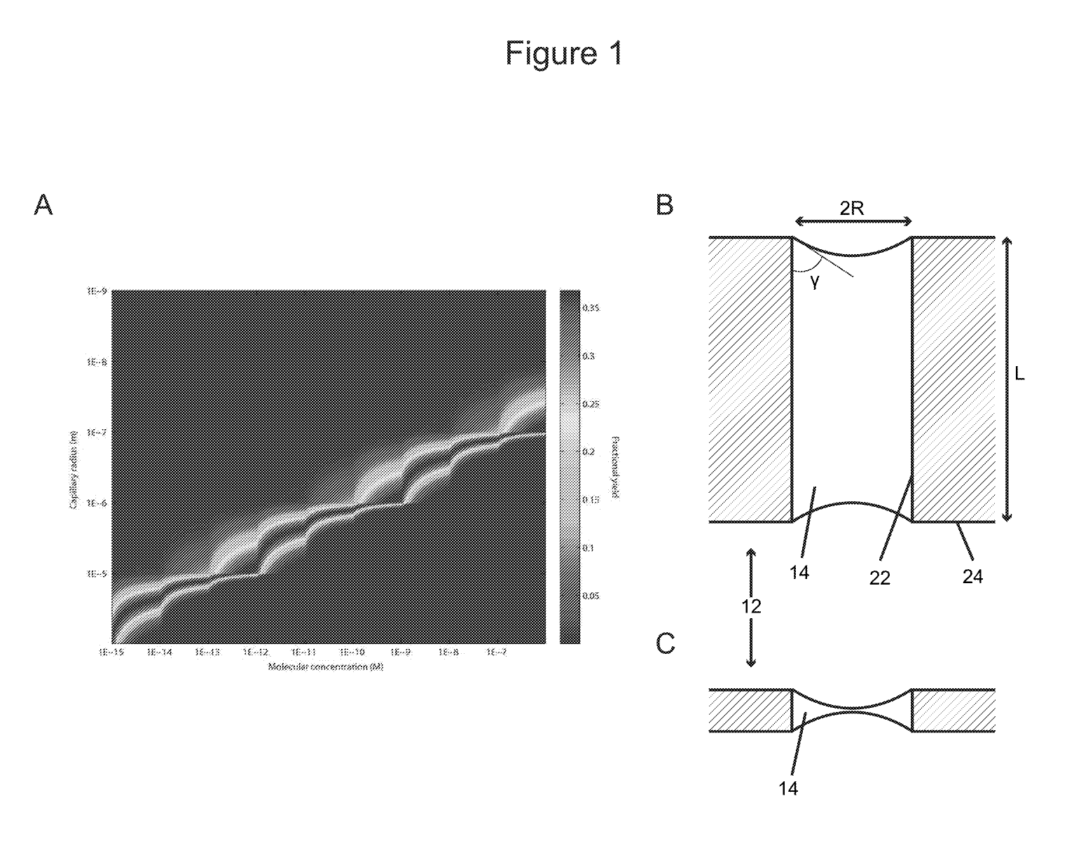

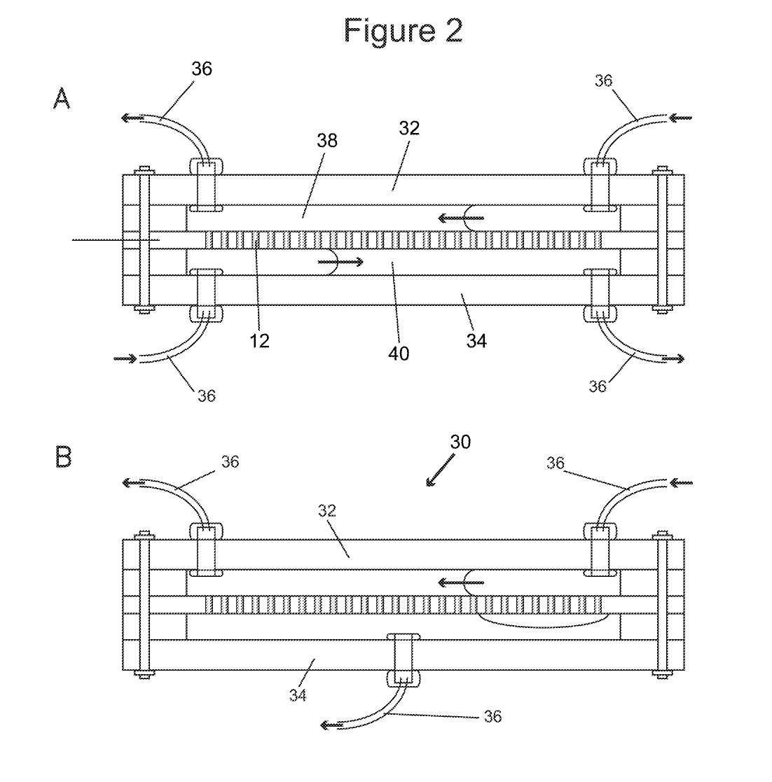

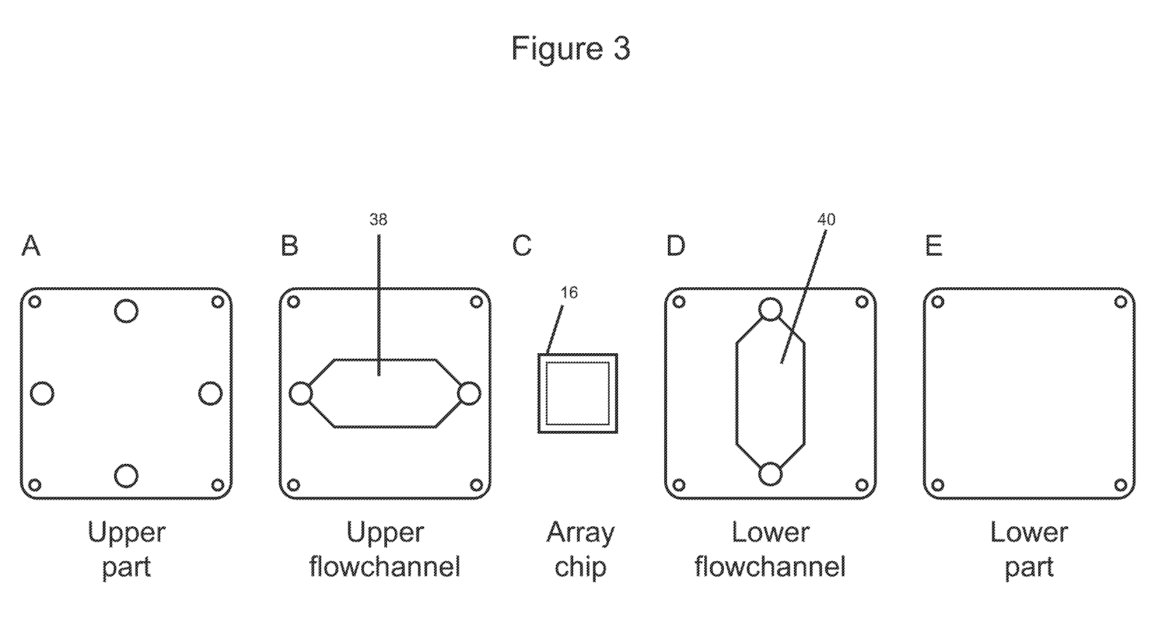

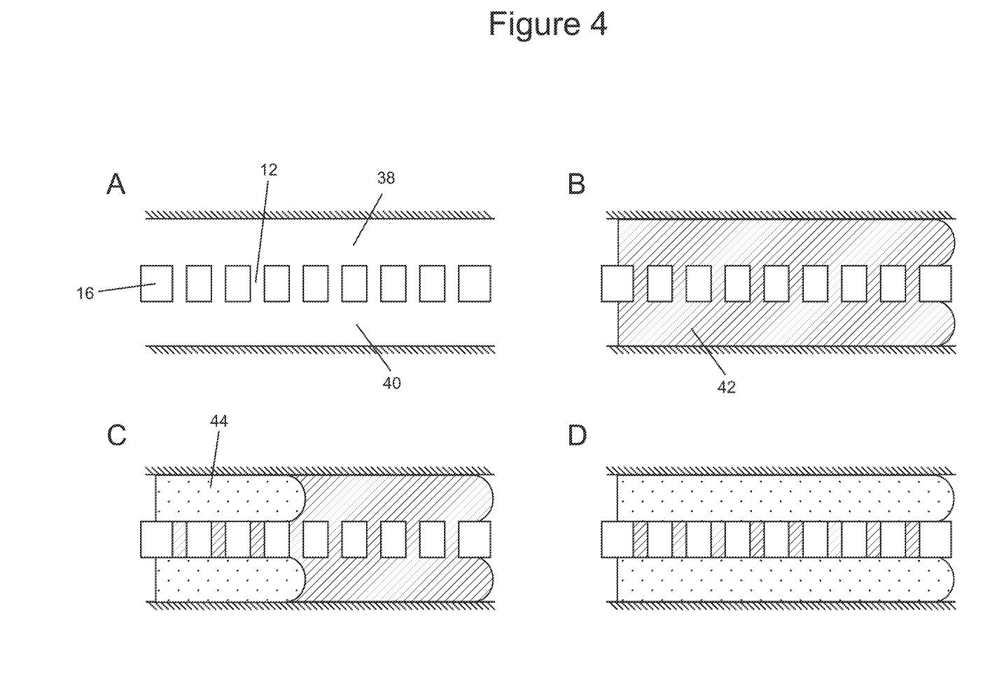

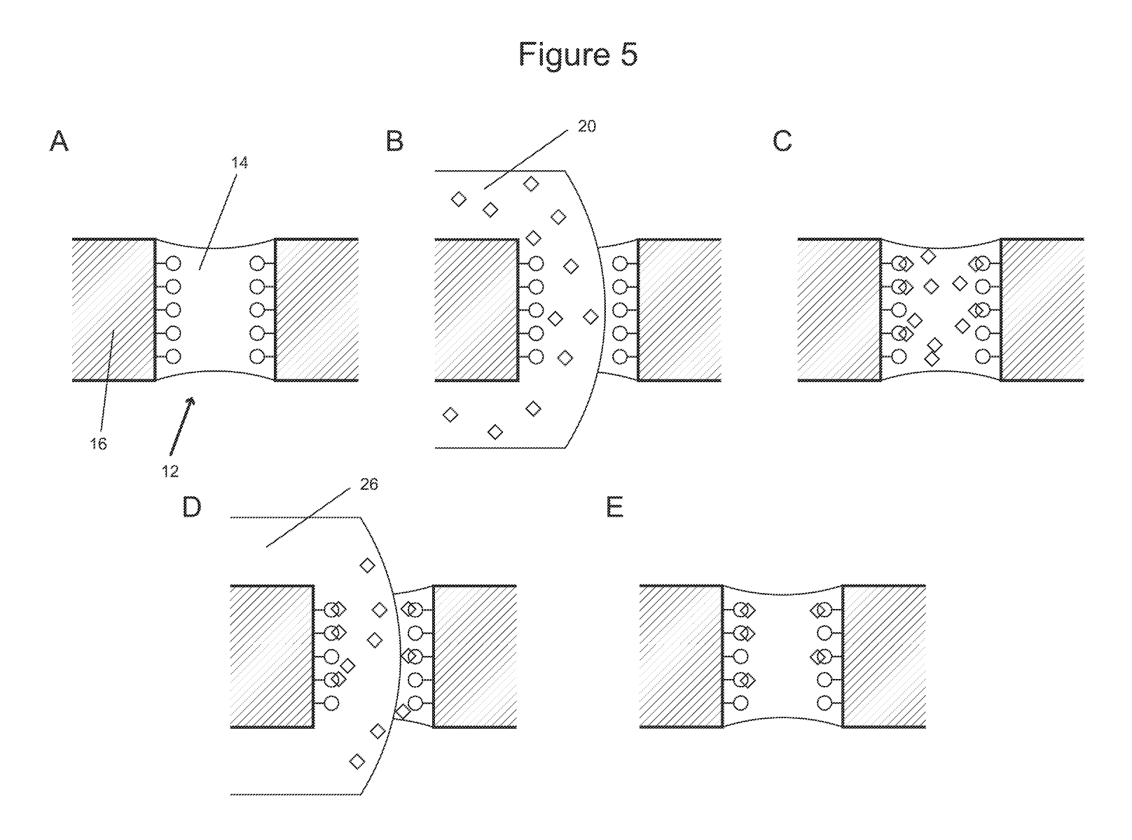

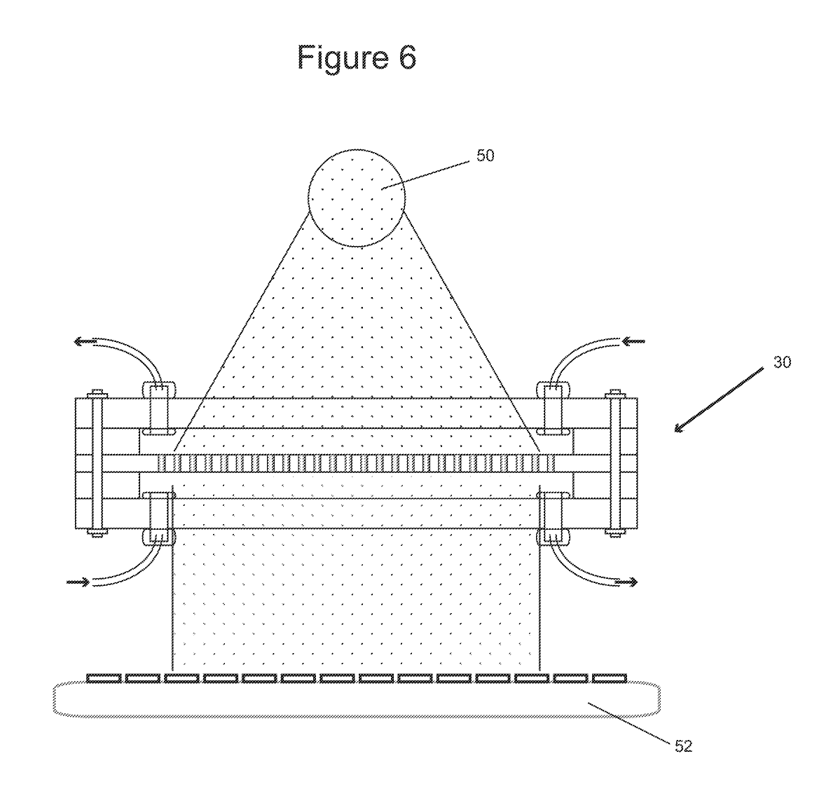

A great variety of optical assays can be conducted in a parallel format using the described array (see FIG. 6). These include measurements of fluorescence intensity, fluorescence polarization, circular dichroism, Förster resonance energy transfer, light absorption, light scattering and luminescence. Depending on the applied detection system the aforementioned measurements may be conducted at deep ultraviolet, ultraviolet, visible, near-infrared, mid-infrared and far-infrared wavelengths and the measurements may or may not be temporally resolved.

To enable parallel assaying, the flowsystem containing the array can be placed in between an illumination source 50 and a detection unit 52, preferably an imaging detector. Further optical elements, such as objectives for magnification, pinholes or emission/excitation/polarization filters may be placed between the flowsystem and the detection unit or the flowsystem and the illumination source. The signal resulting from illumination of the array with a proper wavelength is projected onto the imaging unit, thus resolving the response from individual bores. Furthermore, the setup may be supplemented with a scanning module able to move the array and the detection unit relative to each other, such that data from a greater number of individual array elements may be collected in a single measurement.

Alternatively, using appropriate optical elements in front of the illumination source, it may be focused into a spot and used for scanning the array. The signal may be collected either by an imaging detector or by a point source detector, such as a photomultiplier tube or an avalanche photodiode, as is known from confocal laser scanning microscopy.

Catalytic Activity Analysis.

With the aid of an optical analysis, as the ones mentioned above, a great number of assays testing the performance of chemical and biochemical components may be conducted in individual bores of the array. For example, if the array is loaded with oligonucleotides possessing catalytic activity (the ribozyme) against a certain other molecule (the substrate), an assay may be carried out to measure the amount of substrate converted by the ribozyme. The amount of substrate conversion can be measured optically in a great number of ways, as is known to those skilled in the art, either directly or indirectly with the aid of probe component, which generates an optical signal in response to substrate conversion. The assay may be conducted by introducing substrates and accessory components, such as a probe, to an array pre-loaded with the ribozyme. Alternatively, the assay can be conducted by loading the array with all components (substrate, accessory component and ribozyme) and subsequently monitor the optical response.

The catalytic activity may also be tested for other molecules than oligonucleotides, for example catalytically active polypeptides. In this case, the assay may be conducted in a similar way as what is described above for a ribozyme.

Biological Interaction Analysis.

A great number of biological interactions (see FIG. 5) taking place between oligonucleotides, polypeptides, lipids and small molecules interacting with each other in any combination may be carried out inside individual bores. In all cases, the target molecule (the receptor) is attached to the inner surface of the bore and the binding partner (the ligand 20) is introduced to the bore. Next, after a certain amount of time the array is flushed with a liquid 26. If a biological interaction between ligand and receptor took place, both will remain bound inside the bore, whereas if no interaction took place, the ligand will be removed by the flow. To detect the degree of interaction the amount of ligand can be quantitated optically. Alternatively, another liquid containing probe elements may be introduced to the array after the flushing to enable an optical readout from the ligand or the receptor/ligand-complex.

In the foregoing, analysis and loading has been described primarily using particles of biological material. Clearly, the same dosing and loading criteria are equally valid for non-biological particles, such as particles of other materials, particles of potential catalysts or the like, which are desired analyzed or which take part in an analysis to be performed.

Droplet Evaporation.

Consider a liquid droplet situated in a gaseous atmosphere. Depending on the physical/chemical properties of the gas and the liquid, a certain amount of liquid will be able to evaporate from the droplet and become part of the gas-phase. The rate of evaporation is a function of the surface-area of the droplet exposed to the gas, as well as the temperature of the gas. Generally, the greater the temperature, the more liquid can be absorbed into the gas-phase. Even further, the smaller the droplet size gets, the more severe the impact of evaporation will become. For example, an approximate value for the time (t) it takes for a droplet to evaporate completely would be given as the total droplet volume (V) divided by the evaporation rate (k). If the droplet is spherical with radius (R), then t is

$$\begin{matrix}t = \frac{V}{k} \\ = \frac{4 π {R}^{3}}{12 α ( T ) π {R}^{2}} \\ = \frac{R}{3 α ( T )}\end{matrix} \qquad Eqn . 4$$

Here, α(T) is a characteristic constant of the liquid/gas system, which relates evaporation rate to surface area (A) as well as temperature (T), i.e. k=α(T)A. Hence, according to Eqn. 1, small values of R (a small droplet) lead to correspondingly fast evaporation times.

In certain applications, such as the spotting of biological molecules, it is desirable to produce an ordered array of micro-sized liquid droplets on a surface. However, because the stability of the arrayed droplets is limited they will tend to dry out and consequently have to be rehydrated at a later time. This drying/rehydration process can in many cases cause denaturation of the involved biomolecules, hence compromising their performance. In other applications, such as oligonucleotide amplification using polymerase chain reaction it is necessary to repeatedly increase/decrease the temperature to facilitate efficient amplification.

Even further, for all liquid droplet-based applications integrated into a chip-design, it can prove challenging to enable efficient and repeated temperature-cycling without complete or partial evaporation of the liquid droplet. This is due to the fact that the heating element is situated outside the chip and thus all components inside the chip (liquid droplets the gas-phase) is heated to the desired temperature. This is problematic, because upon heating the gas-phase will be able to absorb more liquid from the droplets and hence compromise droplet integrity.

To solve (or at least drastically reduce) this problem, the chip hosting the droplet array may be connected to pump blowing gas across the chip-surfaces. The gas originates from a liquid reservoir, equipped with a heating element, situated outside the chip. Heating of the liquid reservoir will cause a small part of it to evaporate, and thus saturate the gas-phase. In this way, gas drawn from the reservoir will exhibit α(T)-values close to zero, which leads to t→∞, according to Eqn. 1. If the temperature of the reservoir is changed, it will induce the same temperature change of the arrayed droplets on the chip, but while avoiding evaporation.

1. A method of charging a test carrier with a liquid comprising particles, wherein the carrier comprises a slab having a number of through-going bores extending from a first side of the slab to a second, opposite side of the slab, the bores having a radius of R and a depth L, the test carrier comprising:
a first flow channel comprising a first end having a first opening, a third end having a third opening, and a first surface, positioned between the first and third ends and being at least partly defined by the first side of the slab, and
a second flow channel comprising a second end having a second opening, a fourth end having a fourth opening, and a second surface, positioned between the second and fourth ends and being at least partly defined by the second side of the slab,
the method comprising:
adding, via the first flow channel and to the bores of the carrier, a liquid comprising a carrier liquid with a liquid/air contact angle of γ and a concentration C of particles, where the concentration C of particles and the radius R fulfill the equation of:
P₁=πCR²Lexp(−πCR²L) where L≧R cos(γ) and P₁ exceeds 0.1
flowing a first fluid through the first flow channel and
flowing a second fluid through the second channel.

2. A method according to claim 1, wherein the liquid comprises a plurality, m, of different types of particles, each type of particle being present in a concentration Cm, and C being the sum of all Cm's.

3. A method according to claim 1, further comprising the step of, subsequent to the adding step, fixing one or more of the particle(s) in at least one of the bores.

4. A method according to claim 3, further comprising, subsequent to the fixing step, a second step of adding the liquid to the bores.

5. A method of charging a test carrier with a liquid comprising particles, wherein the carrier comprises a slab having a number of through-going bores having a radius of R and a depth L, each of a first plurality of the bores comprising one or more elements each operative to fix a particle to the bore,
the method comprising:
1. adding, to the bores of the carrier, a first liquid comprising a carrier liquid with a liquid/air contact angle of γ and a concentration C of the particles, where the concentration C of particles and the radius R fulfill the equation of:
P₁=πCR²Lexp(−πCR²L) where L≧R cos(γ) and P1 exceeds 0.1,
2. the fixing elements, in each of a second plurality of the bores of the first plurality, fixing one or more of the particle(s) of the liquid added to the pertaining bore,
3. amplifying/multiplying the particle(s) in the second plurality of bore(s),
4. adding the first liquid to at least one of the bores of the first plurality but not being within the second plurality of bores,
5. the fixing elements, in each of a third plurality of the bores not being part of the second plurality of bores, fixing the one or more of the particle(s) of the liquid added to the pertaining bore,
6. amplifying/multiplying the particle(s) in the third plurality of bore(s),

6. A method of using a test carrier, the method comprising:
providing a test carrier according to claim 1,
adding, to the bores, a second liquid comprising one or more first substances,
detecting a reaction between one or more of the one or more first substances and one or more of the particles.

7. A test carrier with a liquid comprising particles, wherein
the carrier comprises a slab having a number of through-going bores, the bores having a radius of R and a depth L,
the liquid comprises a carrier liquid with a liquid/air contact angle of γ and a concentration C of particles,
where the concentration C of particles and the radius R fulfill the equation of:
P₁=πCR²Lexp(−πCR²L) where L≧R cos(γ) and P₁ exceeds 0.1,
the test carrier further comprising a first and a second flow channel, the first flow channel comprising a first end having a first opening and a first surface at least partly defined by a first side of the slab and opening into the bores, the second flow channel comprising a second end having a second opening and a second surface at least partly defined by a second side of the slab and opening into the bores.

8. A test carrier according to claim 7, wherein the bores have a hydrophilic surface and the slab has, in areas in the vicinity of the bores, a hydrophobic surface.

9. A test carrier according to claim 7, further comprising one or more elements operative to fix the particle(s) in the bores.

10. A test carrier according to claim 7, wherein:
the first flow channel further comprises a third end having a third opening, the first surface being positioned between the first and third ends and
the second flow channel comprises a fourth end having a fourth opening, the second surface being positioned between the second and fourth ends.

11. An apparatus for performing an analysis, the apparatus comprising:
a test carrier according to claim 7,
a sample supply operational to feed a sample to the bores of the test carrier,
a supply of first and second fluid operational to feed first and second fluid to the first and second flow channels, respectively, and
an analysis element operational to detect or determine a reaction between one or more of the particle(s) of the bores and the sample.

12. An apparatus according to claim 11, wherein the sample supply comprises a pump for pumping the sample to the bores via the first flow channel and the first opening.

13. An apparatus according to claim 11, wherein the sample is operational to generate, when reacting with predetermined particle(s), an optically detectable result.

14. A method of using a test carrier, the method comprising:
providing a test carrier according to claim 5,
adding, to the bores, a second liquid comprising one or more first substances,
detecting a reaction between one or more of the one or more first substances and one or more of the particles.
